SEQUENCE LISTING (12 sequences)

SEQ ID NO: 1 - DNA, 756 bp, synthetic construct
  source 1..756: mol_type = other DNA
atggcggccccttcatttgattgtgggaagcctcaagtggagccgaagaaatgtcctggaagggttgtaggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaa

SEQ ID NO: 2 - protein, 236 aa, synthetic construct
MAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLGGTDSCQGDSGGPLVCFEKDKYILQGVTSWGLGCARPNKPGVYVRVSRFVTWIEGVMRNN

SEQ ID NO: 3 - DNA, 15 bp, synthetic construct
  source 1..15: mol_type = other DNA
gagcccacacgaaaa

SEQ ID NO: 4 - protein, 5 aa, synthetic construct
EPTRK

SEQ ID NO: 5 - DNA, 24 bp, synthetic construct
  source 1..24: mol_type = other DNA
ttggagaagtccccaaggccttca

SEQ ID NO: 6 - protein, 8 aa, synthetic construct
LEKSPRPS

SEQ ID NO: 7 - DNA, 15 bp, synthetic construct
  source 1..15: mol_type = other DNA
acaaggtttggaatg

SEQ ID NO: 8 - protein, 5 aa, synthetic construct
TRFGM

SEQ ID NO: 9 - DNA, 15 bp, synthetic construct
  source 1..15: mol_type = other DNA
tcttggggtcttggc

SEQ ID NO: 10 - protein, 5 aa, synthetic construct
SWGLG

SEQ ID NO: 11 - DNA, 24 bp, synthetic construct
  source 1..24: mol_type = other DNA
aatggaagagtccaatccaccgaa

SEQ ID NO: 12 - protein, 8 aa, synthetic construct
NGRVQSTE

(19) United States
(12) Patent Application Publication
(10) Pub. No.: US 2025/0090644 A1
(43) Pub. Date: Mar. 20, 2025

(54) E. COLI EXPRESSED RECOMBINANT HUMAN MICROPLASMIN, A PRODUCTION METHOD THEREOF, A PHARMACEUTICAL COMPOSITION AND A KIT COMPRISING THEREOF, A METHOD AND AN APPLICATION OF TREATING THROMBOEMBOLISM RELATED DISEASES BY USING THEREOF

(71) Applicant: SHENZHEN BAY LABORATORY, Shenzhen City (CN)

(72) Inventors: Xinli Henry LIN, Shenzhen City (CN); Yundong WU, Shenzhen City (CN); Zhengchang LU, Shenzhen City (CN)

(21) Appl. No.: 18/899,715

(22) Filed: Sep. 27, 2024

Related U.S. Application Data
(63) Continuation of application No. PCT/CN2023/084665, filed on Mar. 29, 2023.
Application No. PCT/CN2023/084665 is a continuation of application No. 17/709,403, filed on Mar. 30, 2022, now Pat. No. 11,964,004.

(51) Int. Cl. A61K 38/48; A61P 7/02

(52) U.S. Cl. CPC A61K 38/484; A61P 7/02

(57) ABSTRACT

Provided is an E. coli expressed recombinant human microplasmin (E-μPlm), a production method thereof, a pharmaceutical composition and a kit comprising thereof, an application of the pharmaceutical composition and the kit in treating thromboembolism related diseases and a method of treating thromboembolism related diseases by using the pharmaceutical composition and the kit. The zymogen version of the E-μPlm or E-μPlg is unstable and can undergo autoactivation. Moreover, the in vivo half-life of the E-μPlm is significantly shorter than yeast expressed microplasmin (Y-μPlm), which allows the E-μPlm, the pharmaceutical composition and the kit effective in treating thromboembolism diseases but with reduced bleeding side effects.

### CROSS REFERENCE TO RELATED APPLICATIONS

This application is a Continuation of International Patent Application No. PCT/CN2023/084665, filed on Mar. 29, 2023, which is a Continuation of and claims the benefit of U.S. application Ser. No. 17/709,403, filed Mar. 30, 2022 and titled “SHORT IN VIVO HALF-LIFE AND IN VIVO UNSTABLE RECOMBINANT MICROPLASMIN, PHARMACEUTICAL COMPOSITION COMPRISING THEREOF AND METHOD OF TREATING THROMBOEMBOLISM RELATED DISEASES INCLUDING ADMINISTRATION THEREOF”, the contents of which are incorporated herein by reference in its entirety.

### REFERENCE TO SEQUENCE LISTING

The application contains a Sequence Listing which has been submitted electronically in .XML format together with the International Application and is hereby incorporated by reference in its entirety. Said .XML copy, created on Mar. 25, 2023, is named “core-patent2.xml” and is 16.0 bytes in size.

### TECHNICAL FIELD

The present application relates to the technical field of recombinant protein, more specifically relates to an E. coli expressed recombinant human microplasmin, a production method thereof, a pharmaceutical composition and a kit comprising thereof and an application and a method of treating thromboembolism related diseases by using the pharmaceutical composition and the kit.

### BACKGROUND

Despite the dramatic health impact of the COVID-19 pandemic, cardiovascular disease is still the leading cause of death globally, according to AHA. A key to maintaining a healthy cardiovascular system is to keep a hemostatic balance and disturbing the balance in case of pathogenic thromboembolism can cause major fatal diseases, such as ischemic stroke, myocardial infarction, pulmonary embolism, peripheral arterial occlusion, deep vein thrombosis, and other conditions caused by thromboembolism. The pathological thrombi (blood clots) are dissolved into soluble components in vivo by the enzyme plasmin (Plm), a serine protease that is derived from the proenzyme plasminogen (Plg). Plg is incorporated into a clot as it is formed by binding to both fibrin and fibrinogen. Plg is converted to Plm by both tissue-type Plm activator (tPA) and urokinase-type Plm activator (uPA), which are specific Plg activators in vivo. Plg activators have been developed into thrombolytic drugs for treating thromboembolism diseases. Currently, in the US, the only proven drug for treating acute ischemic stroke is tPA. However, it is well-known that tPA is not an “ideal thrombolytic drug” for the following reasons. First, the efficacy for treating ischemic stroke is only 10% within 3 hours; Second, significant (2%) fatal bleeding side effects (intracranial hemorrhage) limited the treatment within 4.5 hours. Third, the effectiveness of the Plg activator drugs (PAs, including tPA and uPA) depends on Plg present in the clots. And for certain clots, such as the extended and retracted clots present in long-term thromboembolism such as deep vein thrombosis, during thrombolytic therapy with high doses of tPA or uPA, there is a depletion of Plg that may terminate the efficiency of the thrombolytic drug.

In order to solve the “Plg depletion” problem, it has been proposed that a Plm-based direct thrombolysis may have advantages because Plm can directly dissolve the disease-causing clots. However, Plm is immediately neutralized by α2-antiplasmin (α2-AP) in the blood, with an in vivo half-life of only 0.2 seconds. In reality, recombinant μPlm (the catalytic domain of Plm) produced from P. pastoris (Y-μPlm) has been approved by the US FDA for the treatment of symptomatic vitreomacular adhesion in 2012, but met with hurdles when trying to be developed into a thrombolytic drug to treat ischemic stroke and Acute Peripheral Arterial Occlusion (published in two clinical trials: NCT00123305 and NCT00123292). One of the possible reasons is that when using recombinant μPlm as a direct thrombolytic agent, the drug needs to reach enough concentration (a critical concentration for an individual patient) to overcome α2-AP inhibition before effectively dissolving blood clots. The normal plasma concentration of α2-AP is about 1 μM, therefore over 1 μM of μPlm (the critical concentration) is required to neutralize α2-AP before thrombolytic therapy can be effective. With overwhelmingly increased concentration of μPlm, the risk of fatal bleeding side effect will also be increased. Practically, this difficulty causes a “Plm dilemma”: at low dose (less than neutralizing α2-AP), the drug is not effective; but completely neutralizing α2-AP can cause fatal bleeding side effect in clinical applications. Even in the case of locally catheter-directed delivery, the local concentration of Plm has to reach >1 μM to be therapeutically effective. On the other hand, the “overdosed” Plm may reach distant hemostatic plugs that protect blood vessels from bleeding and dissolve them, causing the bleeding side effects.

μPlm is the catalytic domain of Plm, the native enzyme that dissolves blood clots. Recombinant μPlm produced from a yeast P. pastoris strain is a proven drug for treating an eye disease called symptomatic vitreomacular adhesion, but facing hurdles in developing into a thrombolytic drug to treat more severe illnesses such as acute ischemic stroke. Here the inventor of the present application finds that recombinant μPlm produced from the bacteria E. coli (E-μPlm) has unexpected striking differences from Y-μPlm, despite exactly the same functional primary structure. First, E-μPlg, the zymogen version of E-μPlm, is unstable and can undergo autoactivation, a property never found in any other version of Plg, native or recombinant. Second, the melting temperature of E-μPlg is significantly lower than Y-μPlg. Third, the in vivo half-life of E-μPlm is significantly shorter than Y-μPlm. Fourth, animal studies show that E-μPlm is effective in treating thromboembolism diseases but with reduced bleeding side effects. According to these results, the inventor of the present application proposes a strategy that may overcome the long-standing bleeding problem facing thrombolytic therapy. The strategy is summarized by a “hit and die” theory, which describes an “ideal thrombolytic drug”. The “ideal drug” can be delivered to hit a disease-causing thrombus to effectively dissolve it but die out right after because of a short in vivo half-life. As a result, the often-fatal bleeding side effect resulting from the continued proteolytic activity of the present thrombolytic drugs can be avoided. Here the inventor of the present application presents evidence that the E-μPlm developed by the inventor of the present invention may be close to an “ideal thrombolytic drug”.

To solve the “Plm dilemma” problem, the inventor of the present application has developed two different strategies described in our previous publication. The first is to overcome the α2-AP inhibition, and the second is to decrease or eliminate the bleeding side effect. For the first strategy, the inventor of the present application has designed methods to screen and select α2-AP escape mutants. Toward this direction, the inventor of the present application used a structure-based protein engineering strategy to increase the catalytic activity of μPlm while resisting α2-AP inhibition. Part of the results has been published and further described in our review article. For the second strategy, the inventor of the present application proposed a “hit and die” approach. Contrary to the conventional approach of developing in vivo more stable, longer half-life thrombolytic therapeutics, the “hit and die” theory describes an “ideal thrombolytic drug”, which can be delivered to hit a disease-causing thrombus to dissolve it effectively, but die out right after because of a short in vivo half-life. As a result, the often-fatal bleeding side effect resulting from the continued proteolytic activity of the present thrombolytic drugs can be avoided. Unlike the yeast expressed Y-μPlg, the E. coli expression system produces μPlg as insoluble inclusion bodies and needs in vitro refolding to produce an active protein. Significant differences exist between in vitro refolded and natively folded proteins, and in general, E. coli produced and refolded recombinant proteins are less stable and have shorter in vivo half-life than eukaryotic cell-produced proteins. Here the inventor of the present application presents evidence that the E-μPlm developed by the inventor of the present application is significantly different from the Y-μPlm, and is closer to an “ideal thrombolytic drug”. First, E-μPlg, the zymogen version of E-μPlm, is unstable and can undergo autoactivation, a property never found in any other version of Plg, including Y-μPlg. Second, the melting temperature of E-μPlg is significantly lower than Y-μPlg. Third, the in vivo half-life of E-μPlm is significantly shorter than Y-μPlm. Fourth, animal studies show that E-μPlm is effective in treating thromboembolism diseases but with reduced bleeding side effects. The differences are surprising because the E-μPlm and Y-μPlm have exactly the same primary structure, and in a conventional wisdom, the primary structure determines the secondary structure, and the secondary structure in turn determines the tertiary structure and the function of a protein.

### SUMMARY

The present disclosure provides an E. coli expressed recombinant human microplasmin, wherein surface residues of the catalytic domain in contact with the α2-AP in a human microplasmin are changed individually with an Alanine scanning mutagenesis method to enable the E. coli expressed recombinant human microplasmin to dissolve a disease causing thrombus while avoiding α2-AP inhibition and bleeding side effects.

In some embodiments, the surface residues of the catalytic domain that are changed with the Alanine scanning mutagenesis in the E. coli expressed recombinant human microplasmin are respectively shown as loops 1-6, among them
a nucleotide sequence of the loop 1 is shown as SEQ ID NO: 3 and an amino acid sequence of the loop 1 is shown as SEQ ID NO: 4;
a nucleotide sequence of the loop 2 is shown as SEQ ID NO: 5 and an amino acid sequence of the loop 2 is shown as SEQ ID NO: 6;
a nucleotide sequence of the loop 3 is shown as SEQ ID NO: 7 and an amino acid sequence of the loop 3 is shown as SEQ ID NO: 8;
a nucleotide sequence of the loop 4 is shown as cagggtgac and an amino acid sequence of the loop 4 is shown as QGD;
a nucleotide sequence of the loop 5 is shown as SEQ ID NO: 9 and an amino acid sequence of the loop 5 is shown as SEQ ID NO: 10;
a nucleotide sequence of the loop 6 is shown as SEQ ID NO: 11 and an amino acid sequence of the loop 6 is shown as SEQ ID NO: 12.

In some embodiments, the E. coli expressed recombinant human microplasmin is respectively marked by the surface residues of the catalytic domain after mutation as: Gly739Ala, Arg582Ala, Met585Ala, Lys607Ala, Phe587Ala, Ser608Ala, Arg610Ala, Glu641Ala, Pro642Ala.

In some embodiments, a microplasminogen of the E. coli expressed recombinant human microplasmin is autoactivated around pH 6.5.

In some embodiments, an in vivo half-life of the E. coli expressed recombinant human microplasmin is about 14.3±1.6 to about 28.5±1.1 minutes.

In some embodiments, the E. coli expressed recombinant human microplasmin marked as Phe587Ala has an in vivo half life of about 14.3±1.6 minutes.

In some embodiments, the human recombinant microplasmin is selected to be biologically active in cleaving and detoxifying a pathogenic polypeptide or insoluble fibrin and also resistant to α2-antiplasmin inhibition.

The present application further provides a method of producing the above E. coli expressed recombinant human microplasmin, including the following steps:
(1) performing Alanine scanning mutagenesis on the surface residues of the catalytic domain of a human microplasminogen to obtain an exogenous gene of the above E. coli expressed recombinant human microplasminogen;
(2) inserting the exogenous gene into in an E. coli expression system for expression to obtain an insoluble inclusion body of the above E. coli expressed recombinant human microplasminogen;
(3) refolding and purifying the insoluble inclusion body in vitro to obtain mutants of the above E. coli expressed recombinant human microplasminogen;
(4) activating the above E. coli expressed recombinant human microplasminogen into microplasmin by a plasminogen activator such as uPA;
(5) selecting the mutants that are biologically active in cleaving and detoxifying a pathogenic polypeptide or insoluble fibrin and also resistant to α2-antiplasmin inhibition to obtain the above E. coli expressed recombinant human microplasmin.

The present application further provides a pharmaceutical composition comprising the above E. coli expressed recombinant human microplasmin as a thrombolytic agent, or a pharmaceutically acceptable dosage form thereof, or a pharmaceutically acceptable solvate of said compound or dosage form, and including a pharmaceutically acceptable excipient.

The present application further provides a kit comprising the above E. coli expressed recombinant human microplasmin as a thrombolytic agent, or a pharmaceutically acceptable dosage form thereof, or a pharmaceutically acceptable solvate of said compound or dosage form, and including a pharmaceutically acceptable excipient.

The present application further provides an application of the above pharmaceutical composition for the treatment of thromboembolism-related diseases.

The present application further provides an application of the above kit for the treatment of thromboembolism-related diseases.

The present application further provides a method of treating thromboembolism related diseases including ischemic stroke, myocardial infarction, deep vein thrombosis, peripheral arterial occlusion, pulmonary embolism, and systemic blood clotting caused by various disease conditions such as SARS-CoV2 infection and sepsis, wherein the method includes the administration to a subject suffering therefrom a therapeutically effective amount of the above pharmaceutical composition, or a pharmaceutically acceptable dosage form thereof, or a pharmaceutically acceptable solvate of said compound or dosage form.

The present application further provides a method of treating thromboembolism related diseases including ischemic stroke, myocardial infarction, deep vein thrombosis, peripheral arterial occlusion, pulmonary embolism, and systemic blood clotting caused by various disease conditions such as SARS-CoV2 infection and sepsis, wherein the method includes the administration to a subject suffering therefrom a therapeutically effective amount of the above kit, or a pharmaceutically acceptable dosage form thereof, or a pharmaceutically acceptable solvate of said compound or dosage form.

In some embodiments, in the above method of treating thromboembolism related diseases, wherein the pharmaceutical composition is administrated by intravenous, catheter-directed local application, subcutaneous, submuscular, and aerosol routes.

### Definitions

The term “in vivo half-life”, as used herein, means the time it takes for the concentration of a drug in the blood or the amount of a drug in vivo to be decreased to its original half. The calculation formula of in vivo half-life (shown as t1/2) is: t_{1/2}=0.693/k, wherein k is the elimination rate constant. The in vivo half-life of a drug can be calculated according to the above formula as long as the K value of a drug is calculated. For example, if the blood concentration of a drug 2 hours after the administration is 25% and the blood concentration 5 hours after administration is 19%, then the elimination rate constant of this drug, i.e. K=(Inco−Inc)/t=(In25−In19)/(5−2)=0.091 h. The half-life of this drug, i.e. t_{1/2}=0.693/k=7.6 hours.

In the present disclosure, “in vivo half-life” for Plm-based therapeutics has two different definitions. The first definition is the in vivo enzyme activity half-life. Once in the blood, at low concentration (<1 μM, which is the serum concentration of α2-AP), Plm is immediately neutralized by its principle inhibitor α2-AP, and has an activity half-life of only 0.2 seconds, while μPlm has a relatively longer activity plasma half-life of about 4 seconds. The second definition is the inherent structural stability of the protein per se in the blood. For example, recombinant μPlm refolded from E. coli inclusion bodies is stable in the controlled buffer and temperature of laboratory conditions, but the protein itself becomes unstable once injected into the blood of a live mouse, with a structural half-life as short as 2.75 minutes; on the other hand, the in vivo structural half-life of native Plg and Plm can be as long as 2-4 days. In the “hit and die” strategy, the inventor of the present disclosure used the second definition when describing in vivo stability and half-life. In the present disclosure, the “quick death” of the μPlm therapeutics is not resulting from the inhibition of protease inhibitors present in the serum, but is a situation in which at high concentration (>1 μM), after neutralizing all of the inhibitor activities and dissolving the targeting thrombi, the loss of the remaining enzyme activity from the structural disintegration of the recombinant enzyme itself.

The term “recombinant”, as used herein, means that the recombinant vector that can be translated from designed gene into protein fragments and obtained by the gene recombination technology.

The term “refolded”, as used herein, means the process by which an artificially denatured protein acquires its functional structure and conformation. Through this physical process, the protein is folded from random coils into a specific functional three-dimensional structure. When translated from mRNA sequences into linear peptide chains, proteins initially exist as unfolded polypeptides or random coils, and refolded into native conformation by “natural” or “artificial” means.

The term “inclusion body”, as used herein, means the high-density (1.3 mg/ml) insoluble protein particles wrapped by membranes formed when exogenous genes are expressed in prokaryotic cells, especially when they are over-expressed in E. coli, which are present as high refraction area and obviously different from other components in the cytoplast when observed under a microscope. Inclusion bodies generally contain more than 50% of recombinant proteins, wherein the rest are ribosomal elements, RNA polymerase, endotoxin, outer membrane proteins, liposomes, lipopolysaccharides, etc., and are only soluble in denaturing agents such as high concentrations of urea, guanidine hydrochloride, etc. The size of inclusion bodies is about 0.5-1 μm, and the diameter of inclusion bodies in E. coli cytoplasm is generally between 0.2 μm and 1.5 μm.

The term “mutant type”, as used herein, means using genetic engineering method (such as PCR) to introduce mutations in individual amino acid of a native or “WT” proteins.

The term “about” when used immediately preceding a numerical value means±up to 10% of the numerical value. For example, “about 40” means±up to 10% of 40 (i.e., from 36 to 44), ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein.

The term “substantially” has its ordinary meaning as used in the art. For example, “substantially” may mean “significantly” “considerably” “largely” “mostly” or “essentially”. In some embodiments, “substantially” may refer to at least about 50%, at least about 60%, at least about 70%, at least about 80%, at least about 90%, at least about 95%, at least about 96%, at least about 97%, at least about 98%, or at least about 99%.

The term “binding domain” or “binding region” refers to the domain, region, portion, or site of a protein, polypeptide, oligopeptide, or peptide or antibody or binding domain derived from an antibody that possesses the ability to specifically recognize and bind to a target molecule, such as an antigen, ligand, receptor, substrate, or inhibitor. Exemplary binding domains include single-chain antibody variable regions, receptor ectodomains, and ligands. In certain embodiments, the binding domain comprises or consists of an antigen binding site (e.g., comprising a variable heavy chain sequence and variable light chain sequence or three light chain complementary determining regions (CDRs) and three heavy chain CDRs from an antibody placed into alternative framework regions (FRs) (e.g., human FRs optionally comprising one or more amino acid substitutions). A variety of assays are known for identifying binding domains of the present disclosure that specifically bind a particular target, including Western blot, ELISA, phage display library screening, and interaction analysis.

The binding domain or protein “specifically binds” a target if it binds the target with an affinity or Ka (i.e., an equilibrium association constant of a particular binding interaction with units of 1/M) equal to or greater than 105 M1, while not significantly binding other components present in a test sample. Binding domains can be classified as “high affinity” binding domains and “low affinity” binding domains. “High affinity” binding domains refer to those binding domains with a Ka of at least 107 M1, at least 108 M1, at least 109 M1, at least 1010 M1, at least 1011 M1, at least 1012 M1, or at least 1013 M1. “Low affinity” binding domains refer to those binding domains with a Ka of up to 107 M1, up to 106 M1, up to 105 M1. Alternatively, affinity can be defined as an equilibrium dissociation constant (Kd) of a particular binding interaction with units of M (e.g., 105 M to 1013 M). Affinities of binding domain polypeptides and single chain polypeptides according to the present disclosure can be readily determined using conventional techniques.

The term “pharmaceutically acceptable” refers to molecular entities and compositions that do not generally produce allergic or other serious adverse reactions when administered using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The term “pharmaceutically acceptable excipient” means pharmacologically and/or physiologically compatible with the subject and the active ingredient (i.e., capable of eliciting the desired therapeutic effect without causing any adverse desired local or systemic effect), which are well known in the art. Examples of pharmaceutically acceptable excipients include, but are not limited to, fillers, binders, disintegrants, coating agents, adsorbents, anti-adhesive agents, glidants, antioxidants, flavoring agents, coloring agents, Sweeteners, solvents, co-solvents, buffers, chelating agents, surfactants, diluents, wetting agents, preservatives, emulsifiers, coating agents, isotonic agents, absorption delaying agents, stabilizers and tonicity regulators. The selection of suitable excipients is known to those skilled in the art for the preparation of the desired pharmaceutical compositions of the present invention. Exemplary excipients for use in pharmaceutical compositions of the invention include saline, buffered saline, dextrose and water. In general, the selection of suitable excipients depends inter alia on the active agent used, the disease to be treated and the desired dosage form of the pharmaceutical composition.

The terms “nucleic acid,” “nucleic acid molecule,” or “polynucleotide” refer to deoxyribonucleotides or ribonucleotides and polymers thereof in either single-stranded or double-stranded form. Unless specifically limited, the terms encompass nucleic acids containing analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof and complementary sequences as well as the sequence explicitly indicated.

The term “expression” refers to the biosynthesis of a product encoded by a nucleic acid. For example, in the case of nucleic acid segment encoding a polypeptide of interest, expression involves transcription of the nucleic acid segment into mRNA and the translation of mRNA into one or more polypeptides.

The term “E. coli expression system” is an expression system that efficiently expresses genes encoding protective antigens of pathogenic microorganisms in Escherichia coli through DNA recombination technology. Currently, it is one of the most commonly used system for recombinant protein expression. The study of Escherichia coli expression system has many advantages, such as clear background, simple operation, and high expression level. The expression level is significantly higher than that of mammalian expression systems, but there are also corresponding shortcomings, such as the inability of the E. coli system to secrete the expressed protein into the extracellular space, the limited ability to form disulfide bonds, and the inability to achieve complex modifications in protein expression (such as glycosylation modifications).

The terms “polypeptide” “protein” “peptide” or “encoded by a nucleic acid sequence” (i.e., encode by a polynucleotide sequence, encoded by a nucleotide sequence) refer to full-length native sequences, as with naturally occurring proteins, as well as functional subsequences, modified forms or sequence variants so long as the subsequence, modified form or variant retains some degree of functionality of the native full-length protein. In methods and uses of the disclosure, such polypeptides, proteins and peptides encoded by the nucleic acid sequences can be but are not required to be identical to the endogenous protein that is defective, or whose expression is insufficient, or deficient in a subject treated with gene therapy.

The term “subject” refers to an organism, for example, a mammal (e.g., a human, a non-human mammal, a non-human primate, a primate, a laboratory animal, a mouse, a rat, a hamster, a gerbil, a cat, and a dog). In some embodiments, a subject is a mouse. In some embodiments, a human subject is an adult, adolescent, or pediatric subject. In some embodiments, a subject is suffering from a disease, disorder or condition, e.g., a disease, disorder or condition that can be treated as provided herein. In some embodiments, a subject is suffering from a disease, disorder or condition associated with thromboembolism related diseases. In some embodiments, a subject is susceptible to a disease, disorder, or condition. In some embodiments, a susceptible subject is predisposed to and/or shows an increased risk (as compared to the average risk observed in a reference subject or population) of developing a disease, disorder or condition. In some embodiments, a subject displays one or more symptoms of a disease, disorder or condition. In some embodiments, a subject does not display a particular symptom or characteristic of a disease, disorder, or condition. In some embodiments, a subject does not display any symptom or characteristic of a disease, disorder, or condition. In some embodiments, a subject is a human patient. In some embodiments, a subject is an individual to whom diagnosis and/or therapy is and/or has been administered.

The term “substantially” refers to the qualitative condition of exhibition of total or near-total extent or degree of a characteristic or property of interest. One of ordinary skill in the art will understand that biological and chemical phenomena rarely, if ever, go to completion and/or proceed to completeness or achieve or an absolute result. The term “substantially” is therefore used herein to capture the potential lack of completeness inherent in many biological and chemical phenomena.

The term “systemic administration” “administered systemically” “peripheral administration” and “administered peripherally” as used herein have their art-understood meaning referring to administration of a compound or composition such that it enters the recipient's system.

The term “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. The term “WT” has its art-understood meaning that refers to an entity having a structure and/or activity as found in nature in a “normal” (as contrasted with mutant, diseased, altered, etc) state or context. Those of ordinary skill in the art will appreciate that wild type genes and polypeptides often exist in multiple different forms.

The term “treat”, “treatment” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

The term “therapeutically effective amount” refers to an amount that produces the desired therapeutic effect for which it is administered. In some embodiments, the term refers to an amount that is sufficient, when administered to a population suffering from or susceptible to a disease, disorder or condition in accordance with a therapeutic dosing regimen, to treat the disease, disorder or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment.

### ABBREVIATIONS

“AMI” represents Acute Myocardial Infarction; “AD” represents Alzheimer's disease; “α2-AP” represents α2-antiplasmin; “AHA” represents American Heart Association; “CDT” represents Catheter-directed thrombolysis; “μPlm” represents microPlasmin; “Plm” represents plasmin; “Plg” represents plasminogen; “tPA” represents tissue-type plasminogen activator; “uPA” represents urokinase-type plasminogen activator; “mPlg” represents miniplasminogen; “μPlg” represents microplasminogen; “mPlm” represents miniplasmin; “μPlm” represents microplasmin; “PA” represents plasminogen activator; “Kr” represents kringle; “CTT” represents C-terminal tail; “PDB” represents Protein Data Bank; “COVID-19” represents coronavirus disease 2019; “IB” represents inclusion bodies; “M-WT” represents mouse wild type; “H-WT” represents human wild type; “ME-μPlm” represents E. coli expressed mouse microPlasmin; “WT” represents wild type; “M-μPlg” represents mouse microplasminogen; “HY-μPlg” represents yeast expressed human microplasminogen; “MY-μPlg” represents yeast expressed mouse microplasminogen; “H-μPlg” represents human microplasminogen; “Y-μPlg” represents yeast expressed microplasminogen; “HE-μPlg” represents E. coli expressed human microplasminogen; “Y-μPlm” represents yeast expressed microPlasmin; “E-μPlm” represents E. coli expressed microPlasmin; “E-μPlg” represents E. coli expressed microplasminogen; “IC50” represents half maximal inhibitory concentration; “H-WT” represents human wild type; “M-WT” represents mouse wild type; “HE-WT” represents E. coli expressed human wild type; “HE-μPlm” represents E. coli expressed human microPlasmin; “Km” represents Michaelis constant; “Kcat” represents catalytic constant; “Kd” represents dissociation constant; “Vmax” represents maximum speed of response; “H-F587A” represents human Phe587Ala; “F587A” represents Phe587Ala; “R403” represents Arg403; “M404” represents Met404; “H603” represents His603; “D646” represents Asp646; “S741” represents Ser741; “D735” represents Asp735; “S735” represents Ser735; “L763” represents Leu763; “G764” represents Gly764; “S736” represents Ser736; “P642A” represents Pro642Ala; “G739A” represents Gly739Ala; “K645A” represents Lys645Ala; “V720A” represents Val720Ala; “E724A” represents Glu724Ala; “F587R” represents Phe587Arg; “G739A” represents Gly739Ala; “R582A” represents Arg582Ala; “M585A” represents Met585Ala; “K607A” represents Lys607Ala; “S608A” represents Ser608Ala; “R610A” represents Arg610Ala; “E641A” represents Glu641Ala; “G739” represents Gly739; “F587” represents Phe587; “N717A” represents Asn717Ala; “G718A” represents Gly718Ala; “R719A” represents Arg719Ala; “V720A” represents Val720Ala; “Q721A” represents Gln721Ala; “S722A” represents Ser722Ala; “T723A” represents Thr723Ala; “E724A” represents Glu724Ala; “L605A” represents Leu605Ala; “E606A” represents Glu606Ala; “P609A” represents Pro609Ala; “P611A” represents Pro611Ala; “S612A” represents Ser612Ala; “T581A” represents Thr581Ala; “F583A” represents Phe583Ala; “G584A” represents Gly584Ala; “H586A” represents His586Ala; “Q738A” represents Gln738Ala; “D740A” represents Asp740Ala; “S760A” represents Ser760Ala; “W761A” represents Trp761Ala; “G762A” represents Gly762Ala; “L763A” represents Leu763Ala; “G764A” represents Gly764Ala; “P642A” represents Pro642Ala; “T643A” represents Thr643Ala; “R644A” represents Arg644Ala; “H621A” represents His621Ala; “Q622A” represents Gln622Ala; “E623A” represents Glu623 Ala; “V624A” represents Val624Ala; “N625A” represents Asn625Ala; “L626A” represents Leu626Ala; “E627A” represents Glu627Ala; “P628A” represents Pro628Ala; “H629A” represents His629Ala; “T688A” represents Thr688Ala; “Q689A” represent Gln689Ala; “G690A” represents Gly690Ala; “T691A” represents Thr691Ala; “F692A” represents Phe692Ala; “G693A” represents Gly693Ala; “G695A” represents Gly695Ala; “L696A” represents Leu696Ala; “F785C” represents Phe587Cys; “F785D” represents Phe587Asp; “F785E” represents Phe587Glu; “F785G” represents Phe587Gly; “F785H” represents Phe587His; “F785I” represents Phe587Ile; “F785K” represents Phe587Lys; “F785L” represents Phe587Leu; “F785M” represents Phe587Met; “F785N” represents Phe587Asn; “F785P” represents Phe587Pro; “F785Q” represents Phe587Gln; “F785R” represents Phe587Arg; “F785S” represents Phe587Ser; “F785T” represents Phe587Thr; “F785V” represents Phe587Val; “F785W” represents Phe587Trp; “F785Y” represents Phe587Tyr.

### BRIEF DESCRIPTION OF THE DRAWINGS

FIG. 1 shows nucleotide and amino acid sequence of loops in the designed mutant Human μPlg known in the art, respective sequences can be described as:

cDNA sequence of the native human μPlg can refer to SEQ ID NO: 1; amino acid sequence of the native human μPlg can refer to SEQ ID NO: 2;

cDNA sequence of Loop 1 in mutative Human μPlg can refer to SEQ ID NO: 3, wherein the cDNA starts from g298 to a312; amino acid sequence of Loop 1 in mutative Human μPlg can refer to SEQ ID NO: 4, wherein the amino acid starts from Glu1 to Lys5;

cDNA sequence of Loop 2 in native Human μPlg can refer to SEQ ID NO: 5, wherein the cDNA starts from t190 to a213; amino acid sequence of Loop 2 in mutative Human μPlg can refer to SEQ ID NO: 6, wherein the amino acid starts from Leu1 to Pro8;

cDNA sequence of Loop 3 in mutative Human μPlg can refer to SEQ ID NO: 7, wherein the cDNA starts from a118 to g132; amino acid sequence of Loop 3 in mutative Human μPlg can refer to SEQ ID NO: 8, wherein the amino acid starts from Thr1 to Met5;

cDNA sequence of Loop 4 in mutative Human μPlg is cagggtgac; amino acid sequence of Loop 4 in mutative Human μPlg is QGD;

cDNA sequence of Loop 5 in mutative Human μPlg can refer to SEQ ID NO: 9, wherein the cDNA starts from t655 to t669; amino acid sequence of Loop 5 in mutative Human μPlg can refer to SEQ ID NO: 10, wherein the amino acid starts from Ser1 to Gly5;

cDNA sequence of Loop 6 in mutative Human μPlg can refer to SEQ ID NO: 11, wherein the cDNA starts from a526 to a549; amino acid sequence of Loop 6 in mutative Human μPlg can refer to SEQ ID NO: 12, wherein the amino acid starts from Asn1 to Glu8.

FIG. 2 shows detailed view of the active site, wherein the residues S741, H603 and D646 form the catalytic triad to initiate the covalent reaction with the backbone amide between R403 and M404 of α2-AP.

FIG. 3 shows stabilization of the active site by extensive hydrogen bonds with the side chain of key residue R403 of α2-AP.

FIG. 4 shows the example of Michealis-Menten kinetic measurement of WT μPlm.

FIG. 5 shows the comparison of α2-AP inhibition of WT and F587A, shows the reduced inhibition of F587A by α2-AP.

FIG. 6 shows the Kinetic parameters and α2-AP inhibition of loop 1 as increased fold of specific activity and IC50 to α2-AP, wherein 1 represents fold of catalytic efficiency and 2 represents fold of IC50 relative to the WT.

FIG. 7 shows the Kinetic parameters and α2-AP inhibition of loop 2 as increased fold of specific activity and IC50 to α2-AP, wherein 1 represents fold of catalytic efficiency and 2 represents fold of IC50 relative to the WT.

FIG. 8 shows the Kinetic parameters and α2-AP inhibition of loop 3 as increased fold of specific activity and IC50 to α2-AP, wherein 1 represents fold of catalytic efficiency and 2 represents fold of IC50 relative to the WT.

FIG. 9 shows the Kinetic parameters and α2-AP inhibition of loops 4 and 5 as increased fold of specific activity and IC50 to α2-AP, wherein 1 represents fold of catalytic efficiency and 2 represents fold of IC50 relative to the WT.

FIG. 10 shows the Kinetic parameters and α2-AP inhibition of loop 6 as increased fold of specific activity and IC50 to α2-AP, wherein 1 represents fold of catalytic efficiency and 2 represents fold of IC50 relative to the WT.

FIG. 11 shows the Kinetic parameters and α2-AP inhibition of loop 7 as increased fold of specific activity and IC50 to α2-AP, wherein 1 represents fold of catalytic efficiency and 2 represents fold of IC50 relative to the WT.

FIG. 12 shows the Kinetic parameters and α2-AP inhibition of loop 8 as increased fold of specific activity and IC50 to α2-AP, wherein 1 represents fold of catalytic efficiency and 2 represents fold of IC50 relative to the WT.

FIG. 13 shows the Kinetic parameters and α2-AP inhibition of F587 mutants as increased fold of specific activity and IC50 to α2-AP, here present the results of saturation mutation at the 587 position, wherein 1 represents fold of catalytic efficiency and 2 represents fold of IC50 relative to the WT.

FIG. 14 shows a summary of the calculation results of in vivo half life of E-μPlm/Y-μPlm.

FIG. 15 shows the calculation results of in vivo half life of E. coli expressed H-F587A μPlm.

FIG. 16 shows the calculation results of in vivo half life of E. coli expressed E Human-WT μPlm.

FIG. 17 shows mutational mapping onto the interface where α2-AP contacts with surface residue of catalytic domain, wherein interactive locations of 9 mutants on the μPlm with α2-AP are shown in FIG. 17A and locations and escaping indexes of 9 mutants are respectively marked out in FIG. 17B.

FIG. 18 shows SDS-PAGE of purified μPlg and μPlm variants, wherein (A) represents E-μPlg purification by superdex 200, 1: marker, 2: M-WT non-reducing; 3: M-WT reducing; 4: H-WT non-reducing; 5: H-WT reducing; 6: H-F587A non-reducing; 7: H-F587A reducing; (B) represents E-μPlg purification by SP XL, wherein 1: Marker, 2: M-WT non-reducing; 3: M-WT reducing; 4: H-WT non-reducing; 5: H-WT reducing; 6: H-F587A non-reducing; 7: H-F587A reducing; (C) represents E-μPlm purification by SP XL, wherein 1: F587A marker, 2: M-WT non-reducing; 3: M-WT reducing; 4 H-WT non-reducing; 5: H-WT reducing; 6: H-F587A non-reducing; 7: H-F587A reducing; (D) represents Mouse Y-μPlm (MY-μPlm) expression and purification by affinity column gel—(1) left and SP cation exchange column gel—(2) right; (E) represents SDS-PAGE, direct comparison of SP-column purified MY-μPlm (gel 1, left) and HY-μPlm (gel 2, right), in the figure, the arrow means that after activation, μPlm was cut into two molecules, but connected with a disulfide bond. As a result, the non-reduced lane still show a whole molecule, but reduced lane showed two smaller molecules.

FIG. 19 shows μPlg and μPlm purification by Superdex 200 SEC and SP XL cation exchange column chromatography. (a) H-WT μPlg purification by Superdex 200; (b) M-WT μPlg purification by superdex 200; (c) H-F587A μPlg purification by Superdex 200; (d) H-WT μPlg purification by SP XL; (e) M-WT μPlg purification by SP XL; (f) H-F587A μPlg purification by SP XL; (g) H-WT μPlm purification by SP XL; (h) M-WT μPlm purification by SP XL; (i) H-F587A μPlm purification by SP XL, H-WT means human wild type; M-WT means mouse wild type; F587A, human means F587A mutant.

FIG. 20 shows illustration of a typical animal test and results, wherein in the tissue sections, Top panel (I), lung slides: left, thrombin causes blood clots; middle, control, no thrombin, no blood clots; right, thrombin plus ME-μPlm, no blood clots. Lower panel (II), heart sections: left, mouse dead of clotting by thrombin clotting, no blood in the heart; middle, control, no thrombin, normal heart; right, thrombin plus ME-μPlm, normal heart.

### DETAILED DESCRIPTION

The foregoing written specification is considered to be sufficient to enable one skilled in the art to practice the disclosure. The foregoing description and Examples detail certain exemplary embodiments of the disclosure. It will be appreciated, however, that no matter how detailed the foregoing may appear in text, the disclosure may be practiced in many ways and the disclosure should be construed in accordance with the appended claims and any equivalents thereof.

All references cited herein, including patents, patent applications, papers, text books, and the like, and the references cited therein, to the extent that they are not already, are hereby incorporated herein by reference in their entirety.

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### E. coli Expressed Recombinant H-μPlm

As set forth, the invention describes a concept and product that in developing Plm-based therapeutics, such as μPlm-based thrombolytic, it is desirable to design a therapeutics to escape or overcome inhibition by α2-AP while being effective in dissolving disease-causing blood clots, but also lost activity after therapeutic actions, because of instability in vivo and short in vivo half-life, avoiding bleeding side effects. The concept and product is “novel” because the conventional view is that in developing protein therapeutics including thrombolytics, more in vivo stability and longer in vivo half-life is always desirable.

Therefore, the first aspect of the present invention provides an E. coli expressed recombinant human μPlm, which can be just in vivo unstable in a right degree of activity to be long enough to dissolve disease-causing blood clots but not long enough to dissolve the protecting hemostatic plugs, wherein the present disclosure also disclose that some of the mutant types can be characterized to be better or to be almost the same with the wild type. Given this, the in vivo half-life of the recombinant μPlm of the present disclosure is designed and chosen to be in the range from about 14.3±1.6 minutes to about 22.3±1.1 minutes, which is scope long enough to dissolve disease-causing blood clots, but short enough to lost activity after therapeutic actions, thereby avoiding bleeding side effects.

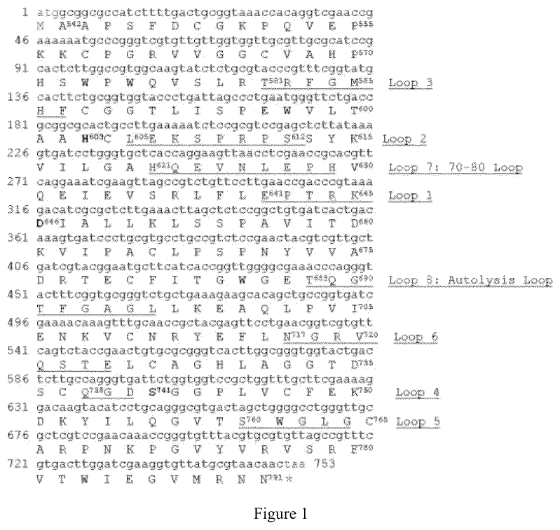

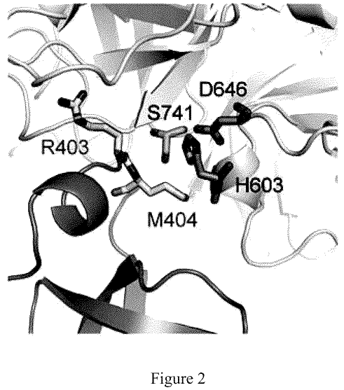

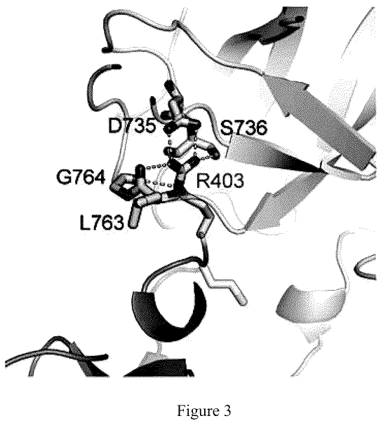

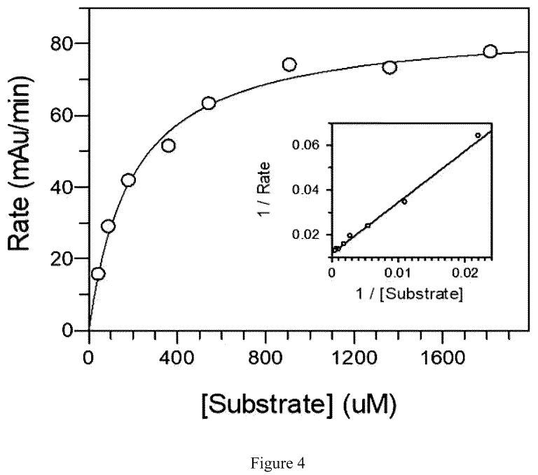

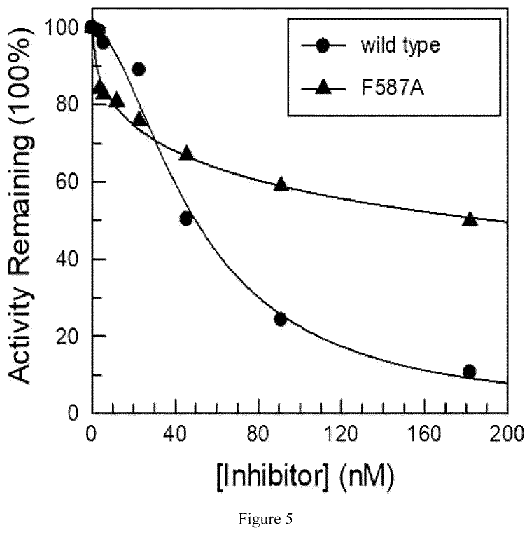

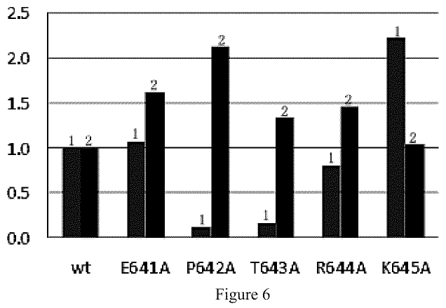

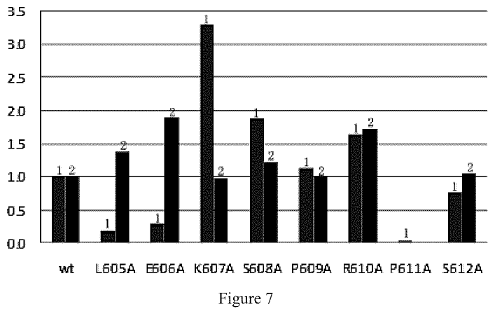

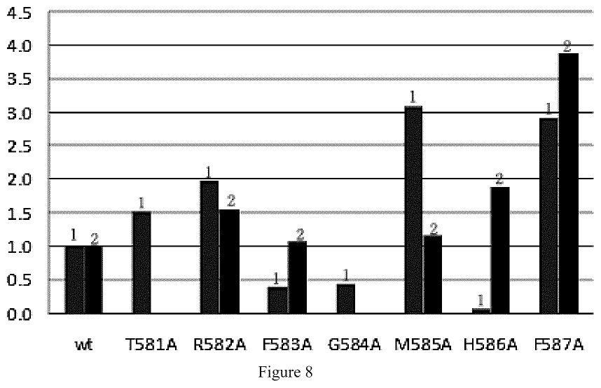

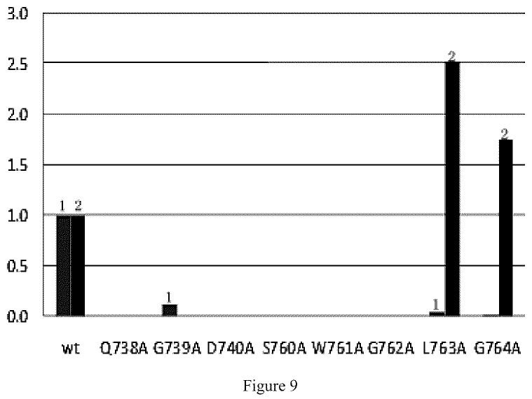

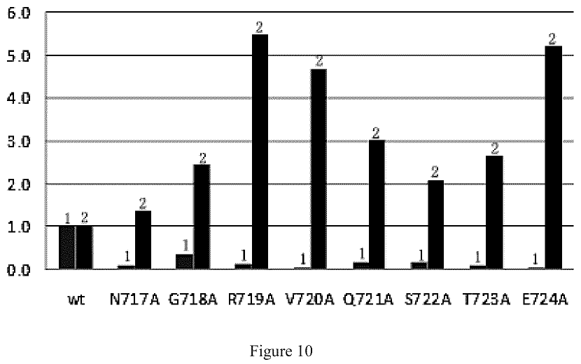

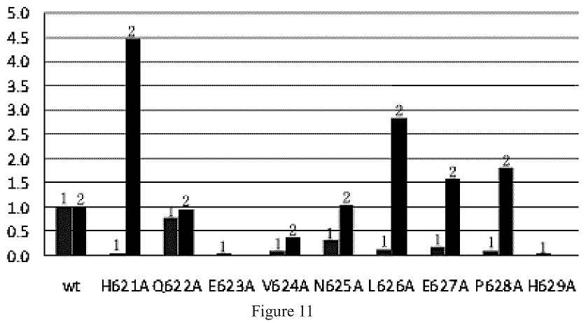

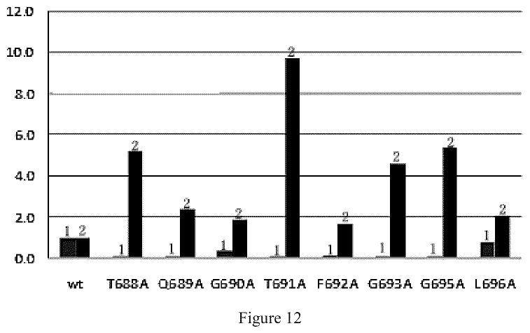

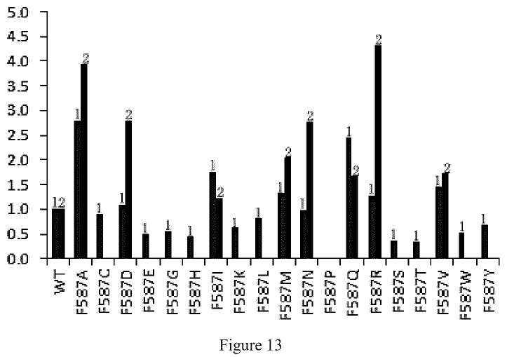

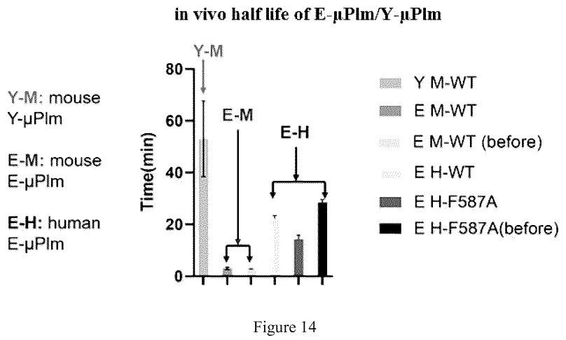

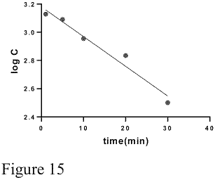

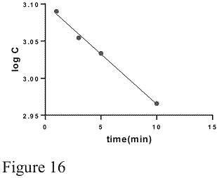

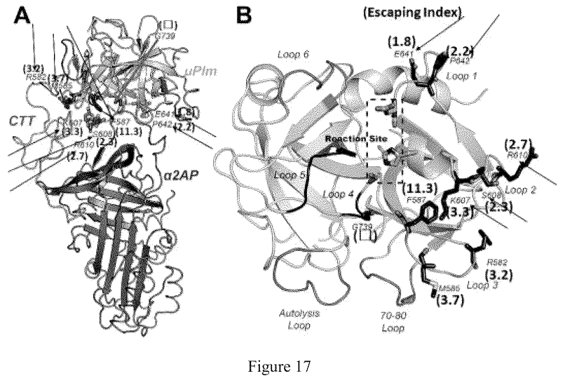

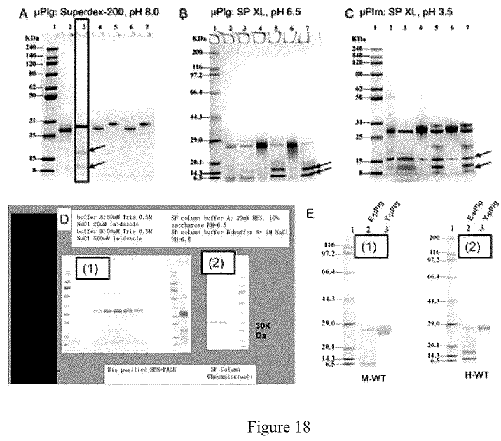

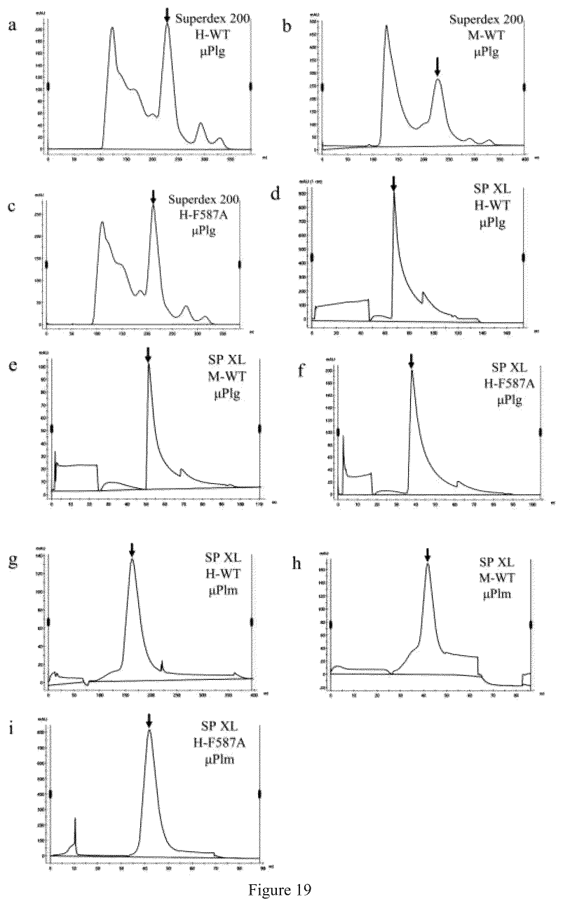

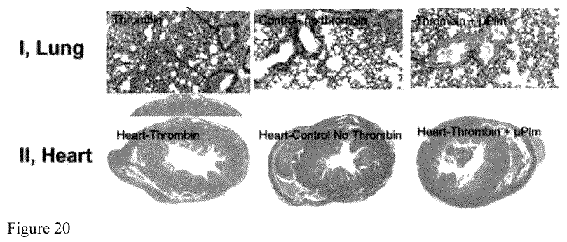

The nucleotide sequence and amino acid sequence of natural human μPlm is shown in FIG. 1, whose nucleotide sequences and amino acid sequences are shown as SEQ ID NO: 1-2, wherein surface residues of the catalytic domain in contact with the α2-AP in a human μPlm are performed with Alanine scanning mutagenesis to enable the E. coli expressed recombinant human μPlm to dissolve a disease causing thrombus while avoiding bleeding side effects. As shown FIG. 1, the surface residues of the catalytic domain that are mutated with the Alanine scanning mutagenesis method in the E. coli expressed recombinant human Plm are respectively shown as loops 1-6, whose nucleotide sequences and amino acid sequences are shown as SEQ ID NO: 3-12.

The inventor of the present application superimposed the crystal structures for individual proteins to the Trypsin: antitrypsin crystal structure (PDB ID: 1OPH) as a template. FIG. 2 and FIG. 3 illustrate that the interface interactions between α2-AP and μPlg is located at the loop of α2-AP that is docked into the active site of μPlm. FIG. 2 shows that the backbone amide between R403 and M404 of α2-AP is the reaction site, which is in the neighboring of the catalytic triads, H603, D646 and S741 of μPlm, with the distance of S741 to the backbone amide of about 4 Å, indicating a pre-attacking pose. FIG. 3 shows the active site is maintained by an extensive network of hydrogen bonds that include the side chain of R403, D735 and S735 along with the backbone carbonyl groups of L763, G764, and S736.

### Cloning, Expression, Inclusion Body Refolding, and Purification of the E. coli Expressed Recombinant Human μPlm

Synthetic genes of the WT and mutant human μPlm optimized for E. coli expression were the same as U.S. Ser. No. 17/709,403. Procedures for E. coli expression, inclusion body purification, refolding and purification were also the same in the prior art. The sequence verified mutant plasmids were transformed into E. coli strain BL21 (DE3) for expression, refolding, and purification following the same procedure in the prior art. Briefly, E. coli containing the expression plasmids was expressed in a high-density shaker flask auto-induction system. The broth was then spun down and the pellet was washed extensively and put through freeze thaw cycles with lysozyme to purify IB. The purified inclusion bodies were dissolved in an 8 M urea buffer (8 M urea, 0.1 M Tris, 1 mM glycine, 1 mM EDTA, 10 mM-mercaptoethanol, 10 mM dithiothreitol (DTT), 1 mM reduced glutathione (GSH), 0.1 mM oxidized glutathione (GSSG), pH 10.5 with a final concentration of 2 mg/ml). The solution was rapidly diluted into 20 volumes of 20 mM Tris, 0.2 M l-arginine, and pH 10.5. The pH of the solution was slowly adjusted to pH 8 with 6 M HCl. The refolded protein was then concentrated by ultrafiltration, and purified by various types of column chromatography. For initial screening, the inventor of the present application grew 200 ml culture for the WT μPlg and each of the mutants, yielding about 200 mg of highly purified IB for each construct.

Human and mouse α2-AP (HAP and MAP) were refolded in a buffer containing 20 mM Tris, 0.1 mM GSH, 0.01 mM GSSG, pH 9.0, and purified essentially the same as that of the μPlg. Streptokinase and staphylokinase were expressed and purified essentially the same as published (High yielding recombinant Staphylokinase in bacterial expression system-cloning, expression, purification and activity studies).

### Enzyme Assay and Kinetic Measurements of the E. coli Expressed Recombinant Human μPlm

The inventor of the present application firstly performed standard Michaelis-Menten kinetic measurements for the WT and all purified mutants, with examples of WT and F587A mutant shown in FIG. 4 and FIG. 5. The inventor of the present application then performed α2-AP inhibition studies, using the WT enzyme as a reference for every set of experiment. FIG. 4 shows an example of Michealis-Menten kinetic measurement of WT μPlm. An example of the inhibition curve for WT and F587A performed side by side is shown in FIG. 5, which indicates clearly that at high concentration of α2-AP, the WT μPlm lost almost all of its activity, while the F587A mutant was still active, keeping almost half of its activity at the highest concentration of the inhibitor tested.

The whole sets of measurement are shown in FIG. 6-13, in a bar graph: the error bars were not illustrated for clarity. Table 1 shows the kinetic parameters of loop 2 and 3 alanine mutants, showed the increased catalytic efficiency (Kcat/Km) of F587A, which exemplified the parameters measured with standard errors.

TABLE 1

| | | | | Fold of |
|---|---|---|---|---|
| | kcat | Km | kcat/Km | kcat/Km |
| μPlg | (min⁻¹) | (μM) | (μM⁻¹min⁻¹) | increased |
| Wild Type | 441.6 ± 13.3 | 203.6 ± 19.5 | 2.2 | 1.0 |
| Thr581Ala | 399.7 ± 5.8 | 121.2 ± 7.6 | 3.3 | 1.5 |
| Arg582Ala | 882.1 ± 24.7 | 205.5 ± 21.2 | 4.3 | 2.0 |
| Gly584Ala | 146.0 ± 2.6 | 154.8 ± 11.1 | 0.9 | 0.4 |
| Met585Ala | 1037.2 ± 32.4 | 154.4 ± 21.0 | 6.7 | 3.1 |
| Phe587Ala | 1135.2 ± 31.3 | 179.4 ± 15.0 | 6.3 | 2.9 |
| Lys607Ala | 1294.3 ± 49.6 | 180.1 ± 25.8 | 7.2 | 3.3 |
| Ser608Ala | 961.1 ± 19.8 | 234.3 ± 17.6 | 4.1 | 1.9 |
| Pro609Ala | 483.4 ± 7.5 | 197.2 ± 13.1 | 2.5 | 1.1 |
| Pro611Ala | 13.8 ± 0.2 | 223.3 ± 11.4 | 0.1 | 0.0 |
| Gln622Ala | 774.1 ± 26.4 | 452.5 ± 42.4 | 1.7 | 0.8 |

Chromogenic substrate pGlu-Phe-Lys-pNA (S-2403) was used for general μPlm enzyme assay and kinetic studies. For μPlm enzyme assay of the mouse plasma samples for in vivo half-life studies, a more sensitive fluorescent assay kit from Abcam (Plm Activity Assay Kit (Fluorometric) (ab204728) was used according to detailed instructions from the manufacturer. The assay mixture contained 2 μl of plasma, 2 μl of the fluorescent substrate (Suc-Ala-Phe-Lys-AMC), and 96 μl of assay buffer provided by the manufacturer.

The kinetic measurements were essentially the same as U.S. Ser. No. 17/709,403. Chromogenic substrate pGlu-Phe-Lys-pNA (S-2403) was used to monitor the proteolytic activity, and 4-Nitrophenyl 4-guanidino benzoate hydrochloride (pNPGB) was used to titer the active site of μPlm. Briefly, recombinant μPlg zymogens (35.5 μM) were activated with a Plg activator such as urokinase (20:1) at 37° C. for 10 minutes in a reaction mixture containing 25 mM Tris-HCl, pH 7.4, 50 mM NaCl. The active site of the activated μPlm was titrated using pNPGB as described. The activated zymogens were diluted to 5.5 μM, and then 10 μl was mixed with 100 μl of 0.0625 mM, 0.125 mM, 0.25 mM, 0.5 mM, 0.75 mM, 1.0 mM, 1.5 mM, or 2.0 mM of substrate S-2403 in the assay buffer (25 mM Tris-HCl, 50 mM NaCl, pH 7.4). The generation of amylolytic activity was monitored (at 405 nm) at 37° C. in 10-second intervals for 20 minutes using a microplate reader from Thermo Fisher. The data was plotted as velocity vs. substrate using GraFit version 7 (Erithacus Software) and the Vmax and Km of the WT and each mutant μPlm were determined. The catalytic efficiency (Kcat/Km) was calculated according to the active enzyme concentration.

Chromogenic substrate pGlu-Phe-Lys-pNA (S-2403) was from Chromogenix (Sweden). 4-Nitrophenyl 4-guanidinobenzoate hydrochloride (pNPGB) was from Aldrich. NUPAGE 4-12% BT GEL was from Invitrogen. Other chemicals and protein reagents were from SIGMA/Aldrich. Kinetic measurement was performed similarly as described. Briefly, the refolded and purified μPlg zymogens (35.5 μM) were activated with a Plg activator such as urokinase (20:1) at 37° C. for 4 minutes in a reaction mixture containing 25 mM Tris-HCl, pH 7.4, 50 mM NaCl. The active site of the activated μPlm was titrated using pNPGB as described. The activated zymogens were diluted to 5.5 μM, and then 10 μl was mixed with 100 μl of 0.0625 mM, 0.125 mM, 0.25 mM, 0.5 mM, 0.75 mM, 1.0 mM, 1.5 mM, or 2.0 mM of substrate S-2403 in the assay buffer (25 mM Tris-HCl, 50 mM NaCl, pH 7.4). The generation of amidolytic activity was monitored (at 405 nm) at 37° C. in 10 seconds intervals for 20 minutes using SpectraMax 250 microplate reader (Molecular Devices). The data was plotted as velocity vs. substrate using GraFit version 7 (Erithacus Software) and the Vmax and Km of the WT and each mutant μPlm were determined. The catalytic efficiency (Kcat/Km) was calculated according to the active enzyme concentration. The μPlm: α2-AP complex was constructed by homology modeling, starting from the crystal structures for μPlm (PDB code: 1BML) and α2-AP (PDB code: 2R9Y). These two structures are superimposed to the crystal structure of Trypsin: antiTrypsin complex (PDB ID: 1OPH) to form the complex. The crystal structure of 2R9Y misses the C-terminal residues, therefore the inventor of the present application uses I-TASSER server to build the missing residues 465-491. The protein complex was then solvated in a rhombic dodecahedron solvent box of water molecules represented by the TIP3P 56 model, and the size of the simulation unit cell was determined to be at least 10 Å away from any atom of the proteins. Counter ions (K⁺ and Cl⁻) were added to ensure electrostatic neutrality corresponding to an ionic concentration of about150 mM. All protein covalent H-bonds were constrained with the LINCS algorithm and long-range electrostatic interactions are treated with the particle-mesh Ewald method with a real-space cutoff of 10 Å. Parallel simulations are performed simultaneously using GROMACS 4.6 in CHARMM36a1 force fields. The system was minimized using the steepest decent algorithm to remove the bad contacts, and then gradually heated to 300 K at a constant volume over 1 ns, using harmonic restraints with a force constant 1,000 kJ/(mol Å²) on heavy atoms of both proteins and nucleotides. Over the following 5 ns of simulations at constant pressure (1 atm) and temperature (300 K), the restraints were gradually released. The systems were equilibrated for an additional 10 ns without positional restraints. A Parrinello-Rahman barostat was used to keep the pressure constant, while a V-rescale thermostat with a time step of 2 fs was used to keep the temperature constant. The system was simulated for 100 ns, with snapshots recorded every 20 ps.

FIG. 6 shows the inhibition kinetics of Loop 1, corresponding to part of the “94-shunt” loop in μPlm, which is connected to the active site D646. The relative catalytic efficiency (kcat/Km, fold increase to the WT) of P642A is decreased dramatically, but the IC50 value is enhanced by a factor of 2. The low activity of P642A is due to a large increase in Km. Although the relative catalytic efficiency of the K645A mutation is increased by twofold, IC50 remains unchanged.

FIG. 7 shows the inhibition kinetics of Loop 2, which corresponds to part of the “60-loop” in μPlm, extending out of the active site H603. The figure shows that the P610A mutation leads to higher catalytic activity and along with an increase in IC50.

FIG. 8 exhibits the inhibition kinetics of Loop 3, corresponding to the “37-loop” in μPlm. Four of the alanine mutants in loop 3 have higher catalytic activities than that of the WT enzyme. The main goal of this study is to discover mutations that have enhanced catalytic activities, and at the same time, are resistant to or capable of “escaping” α2-AP inhibition. In all of the alanine mutants the inventor of the present application has scanned, the F587A change is the best candidate. The inventor of the present application therefore selects the F587 position for saturation mutagenesis.

FIG. 9 displays the inhibition kinetics of mutations in loops 4-5, which is the “oxyanion stabilizing loop” of μPlm and consists of the “S1 entry frame” loop respectively. Clearly, mutations both in loop 4 and loop 5 resulted in dramatic loss of catalytic activity. G739A shows a lower catalytic efficiency (fold of kcat=0.6, Km=4.9), but is not inhibited by α2-AP up to 400 nM. This finding indicates that additional research may be targeted at this position. The loss of catalytic efficiency of G739A is mainly caused by the increase of Km.

FIG. 10 depicts the inhibition kinetics of amino acid alteration in Loop 6, which terms as the “methionine loop” in μPlm. the inventor of the present application finds that mutations in loop 6 also result in dramatic loss of catalytic activity, which may be attributed to increased Km, especially for V720A (fold of kcat=0.1, Km=3.3) and E724A (fold of kcat=0.1, Km=5.2).

FIG. 11 illustrates the results by mutations of Loop 7, the 70-80 loop of the μPlm structure, which is also called the “Ca²⁺ binding” loop. Loss of catalytic activity is observed on alanine mutations in this loop, despite the fact that loop 7 is far away from the active site.

FIG. 12 features the activity profile of Loop 8, or the “autolysis loop” of μPlm, which are not active upon amino acid mutations, and thus are not prone to α2-AP inhibition.

Finally, the inventor of the present application presents the kinetics of saturation mutations at the Phe 587 position shown in FIG. 13, in which five of the mutants gain catalytic activities and show greater resistance to α2-AP inhibition compare to the wild type. Of these mutants, F587A and F587R are identical to have the desirable properties.

### Thermostability of the E. coli Expressed Recombinant Human μPlm

A fluorescence-based thermal denaturation assay (Differential Scanning Fluorimetry, DSF) was used to measure thermostability. The measurement was performed using PCR tubes in a Bio-Rad CFX96 Real-Time PCR system (USA). A typical 40 μl reaction mixture contains SYPRO Orange 5× dye (from Thermo Fisher Scientific), 2.5 μM, 5 μM, and 10 μM μPlg, respectively, in 20 mM HEPES buffer, pH 7.5. The reaction plate was incubated at 25° C. for 30 min and then heated to 100° C. at 0.5° C. intervals, with a settling time of 30 seconds. The “Scan mode” was set to “FRET”, and the fluorescence counts were plotted against temperature. Fluorescence was measured with excitation at 470 nm and emission at 570 nm.

### In Vivo Half-Life of the E. coli Expressed Recombinant Human μPlm

For measuring the In vivo half-life, the inventor of the present application injected 100 μL of each μPlm sample diluted with PBS into the tail veins of the tested mice. The ocular drops of blood were collected at indicated times, samples were collected into 1 ml centrifuge tubes with anticoagulant, and the resulting plasma was used for measuring μPlm enzyme activity directly, with blood samples collected from mice as control. Summary of 6 independent measurements, shows that the ME-μPlm has the shortest half-life, as in FIG. 14, wherein the summary of in vivo half life is shown in Table 2.

TABLE 2

| μPlm | YM-WT | EM-WT | EH-F587A | EH-WT | EM-WT | EH-F587A |
|---|---|---|---|---|---|---|
| Injection(mg) | 0.4(9 μM) | 0.8(18 μM) | 0.8(18 μM) | 0.8(18 μM) | 0.8(18 μM) | 0.8(18 μM) |
| t_{1/2}(minutes) | 53.02 ± 14.59 | 3.01 ± 0.47 | 14.3 ± 1.61 | 22.29 ± 1.12 | 2.75 ± 0.09 | 28.52 ± 1.05 |

The inventor of the present application specifically measures the in vivo half life of E. coli expressed H-F587A μPlm 0.8 mg as shown in FIG. 15 in contrast with HE-WT μPlm 0.8 mg as shown in FIG. 16, wherein F587A-t1/2: 14.3±1.61 minutes, HE-μPlm-t1/2: 22.29±1.12 minutes, which means E-μPlm F587A may be developed into an “ideal thrombolytic drug”.

### Expression, Purification, and Kinetic Studies of the E. coli Expressed Recombinant Human μPlm

The inventor of the present application described the screening and characterization of a total of 71 μPlm mutant clones, in order to select μPlm variants that can escape a2-AP inhibition, and also have good catalytic activity. Based on the previous results, in the present application, the inventor of the present application focused on the E. coli expression, inclusion body refolding, and purification of three recombinant μPlgs, which including WT H-μPlg, WT M-μPlg, and a selected α2-AP escape mutant of H-μPlg, F587A (H-F587A). In addition, in order to compare the biochemistry and kinetic properties of E. coli and yeast expressed μPlg and μPlm, the inventor of the present application cloned both of the WT H-μPlg and M-μPlg into a yeast expression vector and expressed and purified the yeast-expressed recombinant proteins (HY-μPlg and MY-μPlg).

The E. coli expressed insoluble recombinant μPlm is refolded and purified into an active form for thrombolytic applications.

To experimentally map the contribution of individual residues to the complex formation, the inventor of the present disclosure changed each of the amino acid residues in the μPlm loops into alanine (by means of alanine scanning mutagenesis) and made alanine mutations and of them were expressed in E. coli as inclusion bodies, refolded and purified. From kinetic data of the mutant proteins, the inventor of the present disclosure identified F587A as the most desirable mutant and performed saturation mutagenesis on the F587 position. Interestingly, the α2-AP resistant F587A mutant is consistent with published results showing that the same mutant is resistant to certain active-site small molecule inhibitors. Together the inventor of the present disclosure made a total of 73 mutant clones, 71 of these can be expressed and purified. Table 3 listed the kinetic parameters of the 9 most promising mutants selected from our mutagenesis results. The table summarized the best mutants obtained from the alanine scanning mutagenesis data from reference. All of the kinetic values of the mutants are expressed as relative to the μPlm WT enzyme (WT, set to be 1), which has Kcat=442 (min-1) and Km=204 μM. Kcat/Km is the catalytic efficiency and IC50 is the inhibition of μPlm by a2-AP to half of the maximum activity. In the last column, Kcat/Km×IC50 represents an artificial value to define an “escaping efficiency index”. ∞ means no inhibition.

TABLE 3

| Loops | Mutants | Kcat | Km | Kcat/Km | IC50 | Kcat/Km × IC50 |
|---|---|---|---|---|---|---|
| | WT | 1.0 | 1.0 | 1.0 | 1.0 | 1.0 |
| 1 | E641A | 0.7 | 0.6 | 1.1 | 1.6 | 1.8 |
| | K642A | 1.6 | 0.7 | 2.2 | 1.0 | 2.2 |
| 2 | K607A | 2.9 | 0.9 | 3.3 | 1.0 | 3.3 |
| | S608A | 2.2 | 1.2 | 1.9 | 1.2 | 2.3 |
| | R610A | 1.6 | 1.0 | 1.6 | 1.7 | 2.7 |
| 3 | R582A | 2.0 | 1.0 | 2.0 | 1.6 | 3.2 |
| | M585A | 2.3 | 0.8 | 3.1 | 1.2 | 3.7 |
| | F587A | 2.6 | 0.9 | 2.9 | 3.9 | 11.3 |
| 4 | G739A | 0.6 | 4.9 | 0.1 | ∞ | ∞ |

In the table, a column termed Kcat/Km×IC50 is listed, which the inventor of the present disclosure defined as a mutant “escaping efficiency index”-the higher the index number, the better the mutant. The index number for Fubs587A is 11.3, higher than any other mutants, except G739A, which is out of scale and requires more detailed studies. Each of the mutant residues is labeled in the structures of FIG. 17, which showed that 3 mutants are in contact with the modeled CTT structure (R582A, M585A, K607A) and 5 mutants are in contact with the reactive center loop structure of α2-AP (K607A, S608A, R610A, E641A, P642A). F587A is the most promising escape mutant selected, with catalytic activity and efficiency better than the WT but highly resistant to α2-AP inhibition. The G739 position is very close to the active site S741 and G739A has lower catalytic efficiency toward the synthetic substrate, but kinetic data show that this mutant is completely avoided of inhibition by α2-AP. These results opened new doors for further research toward the therapeutic development of Plm-based drugs. The ultimate goal is to find engineered μPlm therapeutics that will be specific toward degrading a targeted pathogenic peptide substrate, but has no or much lower activity toward other untargeted substrates, through structure-based “directional engineering” of the enzyme.

### Method of Producing the E. coli Expressed Recombinant Human μPlm

The present application also provides a method of producing the E. coli expressed recombinant human μPlm. The method includes the following steps:
(1) performing Alanine scanning mutagenesis on the surface residues of the catalytic domain of a human μPlg to obtain an exogenous gene of the E. coli expressed recombinant human μPlg. Alanine is the chiral amino acid with the shortest side chain among amino acids. By mutating an amino acid residue into alanine, it is possible to investigate whether the function of the mutant protein is lost or changed due to the mutation, thereby locating the amino acid residue that has a critical impact on the function of the protein. For example, one way to study the phosphorylation function of serine/threonine is to mutate T/S to A, making this site unable to be phosphorylated. Glycine has no side chains, and the introduction of glycine is relatively easy to change the configuration of the protein, so glycine is generally not used. (2) inserting the exogenous gene into in an E. coli expression system for expression to obtain an insoluble inclusion body of the E. coli expressed recombinant human μPlm. This step includes the following substeps: (11) E. coli cell and gene validation: whether E. coli has chlamydia infection, its growth status, its transfection testing and the exogenous gene sequence validation; (12) Designing, preparing and screening sgRNA and detecting the positive pool: designing 4-6 sgRNAs, transfecting sgRNA and the donor in different ways, screening of positive pool (resistance, fluorescence, drugs or other pressures) and testing the inserting efficiency of positive pools in E. coli cell; (13) Screening culture mediums of E. coli cell for inserting foreign genes: monoclone amplification of E. coli cell, PCR verification of inserted genes, confirming monoclone genotype of E. coli cell through sanger sequencing and other functional verification; (14) quality testing and delivering of E. coli cell inserted with exogenous genes: chlamydia detection and E. coli cell genotype verification, amplification and freezing.
(2) refolding and purifying the insoluble inclusion body in vitro to obtain mutants of the E. coli expressed recombinant human μPlg. FIG. 18 shows the SDS-PAGE of the column chromatography purified samples. FIG. 18A shows the purity of μPlg purified by the Superdex 200 columns. Samples in Lanes 2, 4, 6 are non-reduced, and 3, 5, 7 are reduced. The non-reduced SDS-PAGE showed a lower molecular weight because the disulfide bonds in the protein molecules are all intact, and the “tethered” molecules are smaller in shape than the extended, reduced conformation. The most interesting result is shown in FIG. 18A, lane 3. This lane is M-μPlg run in a reduced condition, which shows that the M-μPlg is partially autoactivated (arrows indicate two fragments after activation), while human μPlg (H-μPlg and H-F589A) are not activated (lanes 5, 7). The results clearly show that M-μPlg is less stable than H-μPlg. FIG. 18B shows the purity of the SP XL column purified μPlg. In this case, lanes 2, 3, 5, and 7 showed smaller molecular weight, which indicates that μPlg are all activated under this condition (pH 6.5). The SDS-PAGE clearly showed that all versions of the E. coli refolded μPlg were autoactivated to μPlm during the purification process with the SP XL cation exchange column chromatography at pH 6.5. FIG. 18C showed the purity of the SP XL column purified μPlm in reduced (lanes 2, 4, 5) and non-reduced (lanes 3, 5, 7) conditions, which showed a similar pattern as that of FIG. 18B, again indicated that the μPlg in FIG. 18B were all activated. FIG. 18D shows the purification of MY-μPlm expressed in a Pichia yeast expression system. The gels showed that the yeast expressed MY-μPlm is stable either in an affinity purification gel—(1) or an SP XL cation exchange column chromatography at pH 6.5 shown in the gel—(2). In both cases, no autoactivation was observed using the same purification procedures as ME-μPlm. A direct comparison of SP-column purified E-μPlm and Y-μPlm is shown in FIG. 18E, which clearly shows that E-μPlm is autoactivated, while Y-μPlm is stable. In FIG. 18E, the Pichia yeast expressed Y-μPlgs were first purified by affinity chromatography (Ni+-affinity column), took advantage of the His-tag installed at the N-terminus of the recombinant proteins, and the affinity-purified proteins were then further purified using SP XL columns. The present application provides several lines of evidence to prove that E-μPlm is strikingly different from Y-μPlm, and may be developed into an “ideal thrombolytic drug”. FIG. 18 shows that both mouse and human E-μPlg can be autoactivated at pH 6.5, while Y-μPlg is stable and cannot be autoactivated under the same conditions. FIG. 18A also shows that the ME-μPlg can be autoactivated at pH 8, while HE-μPlg cannot be autoactivated under the same condition. FIG. 19 shows the purification profiles of recombinant μPlg with Superdex 200 SEC column chromatography and SP-Sepharose cation exchange chromatography. As described previously, the E. coli expressed inclusion bodies were purified, refolded, and the refolded proteins were first purified using Superdex 200 columns (FIG. 18a, b, c), and the second peaks (refolded peaks, arrows) were further purified using SP XL columns (FIG. 19d, e, f). Further, the purified μPlg were activated with Streptokinase and the activated μPlm were also purified using SP XL columns (FIG. 19g, h, i).
(3) activating the above E. coli expressed recombinant human microplasminogen into microplasmin by a plasminogen activator. The above E. coli expressed recombinant human microplasminogen is an inactive precursor of plasma fibrinolytic enzymes, which are activated by various enzymes such as a tissue activator t-PA, urokinase or at a coagulation contact stage. Moreover, exogenous activators such as streptokinase can also play an active role. Fibrinolytic enzymes degrade fibrin and fibrinogen and maintain the patency of blood vessels and glandular ducts.
(4) selecting the mutants that are biologically active in cleaving and detoxifying a pathogenic polypeptide or insoluble fibrin and also resistant to α2-antiplasmin inhibition to obtain the E. coli expressed recombinant human μPlm. Once a desired residue is identified from the alanine scanning mutagenesis, mutations to change to all other 18 amino acid residues (except wild type and alanine) will be made in the selected position, and further selection will be made to identify the best mutation in the selected position. The second round of saturation mutagenesis is performed at the F587 position. To test the alanine scanning mutagenesis strategy, 54 mutant μPlg expression vectors have been constructed. As an example, the results of testing the expression are presented, refolding, and purification of 10 mutants, along with the WT (WT) μPlg enzyme. The expressed mutants belong to the loop 2 (K607A, S608A, P609a, P611A), loop 3 (T581A, R582A, G584A, M585A, F587A), and 70-80 loop (Q622A). The figures show that the WT and mutant μPlm can be purified with a one-step SEC purification after refolding. This simplified purification procedure allows for the proposed high-throughput screening of μPlm mutants. At equal molar or higher concentrations, α2-AP completely inhibits the activity of the WT μPlm, with a typical “bi-phases” kinetic process. The inhibition kinetics of the mutant F587A, however, is not “typical”. Even at 2 times concentration of α2-AP and long term incubation, there is still about 50% activity remaining for the mutant F587A. The F587A mutant has been identified, which not only has higher specific activity than the WT enzyme, but also can partially escape α2-AP inhibition. It is apparent from the figure that the aromatic ring of the F587 is located near the active site S741, and exposed to the solvent. This residue seems to be one of the contact residues to provide binding energies when α2-AP “approaching” and trying to bind to μPlm, and the mutation of the F587weakened both the kinetics and thermodynamics of the binding interactions.

### Pharmaceutical Composition Comprising the E. coli Expressed Recombinant Human μPlm

The present application provides a pharmaceutical composition comprising the E. coli expressed recombinant human μPlm as a thrombolytic agent, or a pharmaceutically acceptable dosage form thereof, or a pharmaceutically acceptable solvate of said compound or dosage form, and including a pharmaceutically acceptable excipient.

The pharmaceutical composition according to the present application can also be prepared in various forms, such as solid, liquid, gaseous or lyophilized forms, especially ointments, creams, transdermal patches, gels, powders, tablets, solutions, gaseous In the form of aerosols, granules, pills, suspensions, emulsions, capsules, syrups, elixirs, extracts, tinctures or liquid extracts, or in a form especially adapted to the desired method of administration. Processes known in the present invention for the production of medicaments may include, for example, conventional mixing, dissolving, granulating, dragee-making, milling, emulsifying, encapsulating, entrapping or lyophilizing processes. Pharmaceutical compositions comprising immune cells such as those described herein are typically provided in solution, and preferably comprise a pharmaceutically acceptable buffer.

In particular embodiments, the present disclosure provides a pharmaceutical composition, or medicament, for preventing or treating thromboembolism related diseases. In some embodiments, a pharmaceutical composition comprises a modified nucleic acid, a recombinant nucleic acid, a viral vector genome, an expression vector, a host cell and a pharmaceutically acceptable carrier.

In some embodiments, a pharmaceutical composition comprises a therapeutically effective amount of a vector or host cell comprising a modified nucleic acid encoding ASPA which can increase the level of expression and/or the level of activity of ASPA in a cell.

In some embodiments, a pharmaceutical composition comprises a therapeutically effective amount of a vector or host cell comprising a modified, nucleic acid encoding ASPA wherein the composition further comprises a pharmaceutically-acceptable carrier, adjuvant, diluent, excipient and/or other medicinal agents. A pharmaceutically acceptable carrier, adjuvant, diluent, excipient or other medicinal agent is one that is not biologically or otherwise undesirable, e.g., the material may be administered to a subject without causing undesirable biological effects which outweigh the advantageous biological effects of the material.

### Kit Comprising the E. coli Expressed Recombinant Human μPlm

The present application provides a kit comprising the E. coli expressed recombinant human μPlm as a thrombolytic agent, or a pharmaceutically acceptable dosage form thereof, or a pharmaceutically acceptable solvate of said compound or dosage form, and including a pharmaceutically acceptable excipient. The kit can also contain buffer solution. In addition to the above components, the kit may further include instructions. These instructions may exist in multiple forms in the kit, and one or more instructions may exist in the kit. One form of these instructions can be in the form of printing information on suitable media or substrates, such as packaging in a reagent box, printing information on one or more pages of paper in a drug description, and so on. The description may exist on a computer-readable medium such as a magnetic disk, CD, DVD, etc. for information recording. The instructions can exist on a website and can be accessed via the Internet at the delete website. Other suitable methods may exist and be included in the kit. Furthermore, the kit can contain a container which may be an indicative indication given by a government regulatory agency that manufactures, uses, or sells drugs or biological products, which indicates that the government regulatory agency that manufactures, uses, or sells the drug or biological product has approved its use on humans.

The kit can include a certain amount of nano dispersed formulations in the form of a unit dose, such as vials or multiple doses. As a result, in some embodiments, the kit comprises one or more unit doses of nano dispersed formulations. As used herein, the term “unit dose” refers to a physiological discrete unit that is physiologically suitable as a unit dose for use in human and animal subjects, and each unit contains an amount sufficient to produce the desired effect to calculate a predetermined amount of the nano dispersed formulation of the present invention. The amount of a unit dose of the title formulation depends on multiple factors, such as the specific active agent used, the effect to be achieved, and the pharmacodynamics associated with the active agent in the subject. In other embodiments, the kit may include a single multiple dose amount of the formulation.

### Application of the Pharmaceutical Composition and the Kit for the Treatment of Thromboembolism-Related Diseases

The present application provides an application of the pharmaceutical composition for the treatment of thromboembolism-related diseases and an application of the kit for the treatment of thromboembolism-related diseases.

Any suitable pharmaceutically acceptable carrier or excipient can be used in the preparation of a pharmaceutical composition according to the invention.

The pharmaceutical composition is typically sterile, pyrogen-free and stable under the conditions of manufacture and storage. A pharmaceutical composition may be formulated as a solution (e.g., water, saline, dextrose solution, buffered solution, or other pharmaceutically sterile fluid), microemulsion, liposome, or other ordered structure suitable to accommodate a high product concentration. In some embodiments, a pharmaceutical composition comprising a modified nucleic acid, vector genome comprising the modified nucleic acid, host cell of the disclosure is formulated in water or a buffered saline solution. A carrier may be a solvent or dispersion medium containing, for example, water, ethanol, polyol (for example, glycerol, propylene glycol, and liquid polyethylene glycol, and the like), and suitable mixtures thereof. Proper fluidity can be maintained, for example, by use of a coating such as lecithin, by maintenance of a required particle size, in the case of dispersion, and by the use of surfactants. In some embodiments, it may be preferable to include isotonic agents, for example, a sugar, a polyalcohol such as mannitol, sorbitol, or sodium chloride in the composition. Prolonged adsorption of an injectable composition can be brought about by including, in the composition, an agent which delays absorption, e.g., a monostearate salt and gelatin. In some embodiments, a nucleic acid, vector and/or host cell of the disclosure may be administered in a controlled release formulation, for example, in a composition which includes a slow release polymer or other carrier that protects the product against rapid release, including an implant and microencapsulated delivery system.

In some embodiments, the pharmaceutical composition is a parenteral pharmaceutical composition, including a composition suitable for intravenous,. In some embodiments, a pharmaceutical composition comprising E. coli expressed recombinant human μPlm is formulated for administration by ICV injection.

The kit can be provided with packaging material and one or more components therein. The kit typically includes a label or packaging insert including a description of the components or instructions for use in vitro, in vivo or ex vivo, of the components therein. A kit refers to a physical structure that contains one or more components of the kit. Packaging material can maintain the components in a sterile manner and can be made of material commonly used for such purposes (e.g., paper, glass, plastic, foil, ampules, vials, tubes, etc). The label or insert can include identifying information of one or more components therein, dose amounts, and clinical pharmacology of the active ingredients including mechanism of action, pharmacokinetics and pharmacodynamics. A label or insert can include information identifying manufacture, lot numbers, manufacture location and date, expiration dates. A label or insert can include information on a disease for which a kit component may be used. A label or insert can include instructions for a clinician or subject for using one or more of the kit components in a method, use or treatment protocol or therapeutic regimen. Instructions can include dosage amounts, frequency of duration and instructions for practicing any of the methods, uses, treatment protocols or prophylactic or therapeutic regimens described herein. A label or insert can include information on potential adverse side effects, complications or reaction, such as a warning to a subject or clinician regarding situations where it would not be appropriate to use a particular composition.

### Method of Treating Thromboembolism Related Diseases With a Therapeutically Effective Amount of the Pharmaceutical Composition and the Kit

The present application provides a method of treating thromboembolism related diseases including ischemic stroke, myocardial infarction, deep vein thrombosis, peripheral arterial occlusion, pulmonary embolism, and systemic blood clotting caused by various disease conditions such as SARS-CoV2 infection and sepsis, wherein the method includes the administration to a subject suffering therefrom a therapeutically effective amount of the pharmaceutical composition and the kit, or a pharmaceutically acceptable dosage form thereof, or a pharmaceutically acceptable solvate of said compound or dosage form.

In some embodiments, the pharmaceutical composition is administrated by intravenous, catheter-directed local application, subcutaneous, submuscular, and aerosol routes. In animal models, E-μPlm can rescue the mouse from thrombin-induced pulmonary embolism and death, without inducing fatal bleeding side effects. Table 4 shows that in a mouse MCAO model, E-μPlm can completely rescue the mouse from fatal ischemic stroke, without inducing bleeding side effects, while tPA's therapeutic effect is less satisfactory and inducing bleeding side effects. Specific steps of performing animal tests can refer to U.S. Ser. No. 17/709,403.

### Animal Disease Model-1, Pulmonary Embolism Model

Table 4 shows the results of the mouse pulmonary embolism model, wherein shows that in the negative control (lanes 1, 2), no thrombin and drug was injected, and both animals survived. In the positive control (lanes 3, 4), thrombin was injected, but no drug was injected, and animals were dead at 17 and 10 minutes, respectively. In all of the test cases (lanes 5-10), the E-μPlm drug was injected 5 minutes after thrombin injection, resulting in all animals surviving. These results showed that the drug (E-μPlm) could rescue animals from the thrombin-induced fatal pulmonary embolism.

TABLE 4

| | 1 | 2 | 3 | 4 | 5 | 6 | 7 | 8 | 9 | 10 |
|---|---|---|---|---|---|---|---|---|---|---|
| Types | [symbol] | [symbol] | [symbol] | [symbol] | [symbol] | [symbol] | [symbol] | [symbol] | [symbol] | [symbol] |
| | control-1 | control-2 | control-3 | control-4 | control-5 | control-6 | control-7 | control-8 | control-9 | control-10 |
| [symbol] | 42.2 | 42.9 | 36.6 | 45 | 47 | 36.1 | 40.9 | 42.8 | 42.1 | 34.5 |
| [symbol] | 216.9 | 821.75 | 714.6 | [symbol] | 852.6 | 270.76 | [symbol] | 323 | 352.25 | [symbol] |
| [symbol] | 0 | 9 | 73.2 | 90 | 94 | 94 | 81.8 | 85.6 | 94.2 | 69 |
| [symbol] | 85 | 86 | 0 | 0 | 0 | 0 | 0 | 0 | 0 | 0 |
| [symbol] | 0 | 0 | 0 | 0 | 200 | 200 | 200 | 200 | 200 | 200 |
| [symbol] | 0 | 0 | 0 | 0 | 200 | 200 | 200 | 400 | 600 | 600 |
| [symbol] | 0 | 0 | 0 | 0 | 5 | 5 | 5 | 10 | 15 | 15 |
| [symbol] | 5 | 5 | 5 | 5 | 5 | 5 | 5 | 5 | 5 | 5 |
| [symbol] | / | / | 17 | 10 | / | / | / | / | / | / |

[symbol]

indicates data missing or illegible when filed

FIG. 20 shows tissue sections after the experiment. The top panel (I) shows lung tissue sections, which show that thrombin causes blood clots (left panel), the control shows no blood clots (middle panel), and E-μPlm dissolved thrombin-caused blood clots (right panel). The lower panel (II) shows heart tissue sections, which shows that thrombin causes blood clots in the lung, and no blood in the heart (left panel). The control without thrombin shows normal blood in the heart (middle panel), and E-μPlm recured thrombin caused blood blockage, and normal blood in the heart (right panel).

### Animal Disease Model-2, MCAO Model

Table 5 shows that in the mouse MCAO model, mice in the sham group are all normal, but the neurological function of the control group has a clear deficit, with a deficit score as high as 4.3. The E-μPlm group has a significant difference from the control group, with significantly lower neurological deficit scores. The death count shows that in the control group, there are 6 deaths (37.3%), and for the E-μPlm group, in the low dose group (5 mg/kg), there is 1 death only. Even more important, there is no death in the high dose group (10 mg/kg). This is in clear contract with tPA groups: in the low-dose group (4 mg/kg), there were 2 deaths, and in the high-dose group, there were 3 deaths. The higher death rate of the tPA's high dose group is attributed to the bleeding side effect of tPA. In addition, the control group had a high infarct volume (351.3 mm3), but both E-μPlm and tPA treatment greatly reduced the infarct volume, with a high dose of μPlm the greatest contribution.

TABLE 5

| | Dose | Sur- | | | | |
|---|---|---|---|---|---|---|
| | mg/ | vival/ | Death/ | Mortality/ | Neurological | Infarct |
| Group | kg | n | n | % | deficit score | vlume/mm³ |
| Sham | | 10 | 0 | 0.0 | 0.0 ± 0.0 | 0.0 ± 0.0 |
| Control | | 10 | 6 | 37.3 | 4.3 ± 0.8 | 351.3 ± 21.4 |
| E-μPlm | 5 | 10 | 1 | 9.1 | 1.7 ± 0.7 | 142 ± 32.2 |
| tPA | 10 | 10 | 0 | 0.0 | 1.0 ± 0.5 | 73.3 ± 29.7 |
| | 4 | 10 | 2 | 16.7 | 1.8 ± 0.9 | 108.5 ± 23.1 |
| | 8 | 10 | 3 | 23.1 | 3.0 ± 2.1 | 109.5 ± 24.2 |

Table 6 shows that treatment with μPlm resulted in no bleeding time increase, clearly indicating that the μPlm treatment is safe. However, tPA treatment greatly increased bleeding time, indicating that tPA has less safety profile than μPlm.

TABLE 6

| | | Bleeding-time | | | |
|---|---|---|---|---|---|
| | Dosae | Pretreat- | 15 | 30 | 60 |
| Group | mg/kg | ment | minutes | minutes | minutes |
| E-μPlm | 5 | 108 ± 12 | 110 ± 10 | 108 ± 9 | 109 ± 11 |
| | 10 | 109 ± 13 | 113 ± 11 | 114 ± 15 | 116 ± 13 |
| tPA | 4 | 111 ± 15 | 140 ± 12 | 145 ± 17 | 147 ± 15 |
| | 8 | 110 ± 12 | 153 ± 15 | 149 ± 19 | 155 ± 14 |

### Discussion

PAs have dominated thrombolytic therapy, but low efficacy and bleeding side effects problems are inherent and cannot be solved easily. According to Marder's analysis, the future therapeutic direction should be Plm-based direct thrombolysis. Marder indicated that there are two major reasons for choosing Plm over PAs. First, Plm is the enzyme that dissolves fibrin, and Plm-based therapeutics would be more efficient and can avoid the Plg depletion problems in certain clinical situations. Second, with the maturation and advancement of CDT, Plm can be delivered directly into the blocking thrombi, and will be neutralized by α2-AP once diffused into the serum, potentially avoiding the bleeding side effect.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Acts, elements, and features discussed only in connection with one embodiment are not intended to be excluded from a similar role in other embodiments. Further, for the one or more means-plus-function limitations recited, for example, in claimed inventions, if any, the means are not intended to be limited to the means disclosed herein for performing the recited function, but are intended to cover in scope any means, known now or later developed, for performing the recited function.

Use of ordinal terms such as “first”, “second”, “third”, etc., in claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, use of these terms in the specification does not by itself connote any required priority, precedence, or order. Neither does use of any such terms indicate number of elements in described (including claimed). The foregoing written specification is sufficient to enable one skilled in the art to practice any invention described in the present disclosure. The present disclosure is not to be limited in scope by examples provided, which are intended as illustrations of one or more aspects of described inventions and other functionally equivalent embodiments are within the scope of described inventions. Various modifications of described inventions in addition to those shown and described herein will become apparent to those skilled in the art from the foregoing description and fall within the scope of described inventions. Advantages and objects of described inventions are not necessarily encompassed by each embodiment of described inventions.

The foregoing written specification is considered to be sufficient to enable one skilled in the art to practice the invention. The present disclosure is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended Embodiments. The advantages and objects of the invention are not necessarily encompassed by each embodiment of the invention.

Throughout this application, various publications are referenced. The disclosures of these publications in their entireties are hereby incorporated by reference into this application for all purposes.

What is claimed is:

1. An E. coli expressed recombinant human microplasmin, wherein surface residues of the catalytic domain in contact with the α2-Antiplasmin in a human microplasmin are individually changed with an Alanine scanning mutagenesis method to enable the E. coli expressed recombinant human microplasmin to dissolve a disease causing thrombus while avoiding α2-Antiplasmin inhibition and bleeding side effects.

2. The E. coli expressed recombinant human microplasmin of claim 1, wherein the surface residues of the catalytic domain that are changed with the Alanine scanning mutagenesis method in the E. coli expressed recombinant human microplasmin are respectively shown as loops 1-6, among them
a nucleotide sequence of the loop 1 is shown as SEQ ID NO: 3 and an amino acid sequence of the loop 1 is shown as SEQ ID NO: 4;
a nucleotide sequence of the loop 2 is shown as SEQ ID NO: 5 and an amino acid sequence of the loop 2 is shown as SEQ ID NO: 6;
a nucleotide sequence of the loop 3 is shown as SEQ ID NO: 7 and an amino acid sequence of the loop 3 is shown as SEQ ID NO: 8;
a nucleotide sequence of the loop 4 is shown as cagggtgac and an amino acid sequence of the loop 4 is shown as QGD;
a nucleotide sequence of the loop 5 is shown as SEQ ID NO: 9 and an amino acid sequence of the loop 5 is shown as SEQ ID NO: 10;
a nucleotide sequence of the loop 6 is shown as SEQ ID NO: 11 and an amino acid sequence of the loop 6 is shown as SEQ ID NO: 12.

3. The E. coli expressed recombinant human microplasmin of claim 1, wherein the E. coli expressed recombinant human microplasmin is respectively marked by the surface residues of the catalytic domain after mutation as: Gly739Ala, Arg582Ala, Met585Ala, Lys607Ala, Phe587Ala, Ser608Ala, Arg610Ala, Glu641Ala, Pro642Ala.

4. The E. coli expressed recombinant human microplasmin of claim 1, wherein a microplasminogen of the E. coli expressed recombinant human microplasmin is autoactivated around pH6.5.

5. The E. coli expressed recombinant human microplasmin of claim 1, wherein the in vivo half-life of the E. coli expressed recombinant human microplasmin is about 14.3±1.6 minutes to about 28.5±1.1 minutes.

6. The E. coli expressed recombinant human microplasmin of claim 1, wherein the human recombinant microplasmin is selected to be biologically active in cleaving and detoxifying a pathogenic polypeptide or insoluble fibrin and also resistant to α2-antiplasmin inhibition.

7. A method of producing the E. coli expressed recombinant human microplasmin of claim 1, including the following steps:
(1) performing Alanine scanning mutagenesis on the surface residues of the catalytic domain of a human microplasminogen to obtain an exogenous gene of the E. coli expressed recombinant human microplasminogen;
(2) inserting the exogenous gene into in an E. coli expression system for expression to obtain an insoluble inclusion body of the E. coli expressed recombinant human microplasminogen;
(3) refolding the insoluble inclusion body in vitro and purifying the refolded protein to obtain mutants of the E. coli expressed recombinant human microplasminogen;
(4) activating the above E. coli expressed recombinant human microplasminogen into microplasmin by a plasminogen activator;
(5) selecting the mutants that are biologically active in cleaving and detoxifying a pathogenic polypeptide or insoluble fibrin and also resistant to α2-antiplasmin inhibition to obtain the E. coli expressed recombinant human microplasmin of claim 1.

8. A pharmaceutical composition comprising the E. coli expressed recombinant human microplasmin of claim 1 as a thrombolytic agent, or a pharmaceutically acceptable dosage form thereof, or a pharmaceutically acceptable solvate of said compound or dosage form, and including a pharmaceutically acceptable excipient.

9. A kit comprising the E. coli expressed recombinant human microplasmin of claim 1 as a thrombolytic agent, or a pharmaceutically acceptable dosage form thereof, or a pharmaceutically acceptable solvate of said compound or dosage form, and including a pharmaceutically acceptable excipient.

10. An application of the pharmaceutical composition of claim 8 for the treatment of thromboembolism-related diseases.

11. An application of the kit of claim 9 for the treatment of thromboembolism-related diseases.

12. A method of treating thromboembolism related diseases including ischemic stroke, myocardial infarction, deep vein thrombosis, peripheral arterial occlusion, pulmonary embolism, and systemic blood clotting caused by various disease conditions such as SARS-CoV2 infection and sepsis, wherein the method includes the administration to a subject suffering therefrom a therapeutically effective amount of the pharmaceutical composition of claim 8, or a pharmaceutically acceptable dosage form thereof, or a pharmaceutically acceptable solvate of said compound or dosage form.

13. A method of treating thromboembolism related diseases including ischemic stroke, myocardial infarction, deep vein thrombosis, peripheral arterial occlusion, pulmonary embolism, and systemic blood clotting caused by various disease conditions such as SARS-CoV2 infection and sepsis, wherein the method includes the administration to a subject suffering therefrom a therapeutically effective amount of the kit of claim 9, or a pharmaceutically acceptable dosage form thereof, or a pharmaceutically acceptable solvate of said compound or dosage form.

14. The method of treating thromboembolism related diseases of claim 12, wherein the pharmaceutical composition is administrated by intravenous, catheter-directed local application, subcutaneous, submuscular, and aerosol routes.
